(19) United States
(12) Patent Application Publication
(10) Pub. No.: US 2004/0254600 A1
(43) Pub. Date: Dec. 16, 2004

(54) Methods and devices for endovascular mitral valve correction from the left coronary sinus

(76) Inventors: David Zarbatany, Laguna Niguel, CA (US); Stefan Schreck, Vista, CA (US)

Correspondence Address: EDWARDS LIFESCIENCES CORPORATION, ONE EDWARDS WAY, IRVINE, CA, 92614, US

(21) Appl. No.: 10/787,574

(22) Filed: Feb. 25, 2004

Related U.S. Application Data
(60) Provisional application No. 60/449,960, filed on Feb. 26, 2003.

(51) Int. Cl. A61M 29/00

(52) U.S. Cl. 606/194

(57) ABSTRACT

Apparatuses and methods for reshaping a mitral valve annulus to correct for mitral regurgitation. The apparatuses include one or more balloons in a balloon assembly that are delivered in a deflated state and inflated within the left coronary sinus adjacent mitral annulus. The balloon or balloon assembly may be linear and have one or both ends more flexible than a mid-section, or may be curvilinear. A single balloon having differently constructed end sections may be used. Alternatively, a balloon assembly may include two concentrically arranged balloons with an inner, shorter balloon and an outer, longer balloon. The outer balloon defines the ends of the balloon assembly and is inflated to a lesser pressure than the inner balloon so as to result in the flexible ends. Two or more balloons in series may be mounted on a catheter with gaps therebetween to permit relative flexing or bending. The inflation fluid may be saline or other biocompatible inflation fluid, or may be a fluid that can be subsequently hardened by curing or cross-linking. In either case, a one-way valve is typically utilized to prevent deflation of the balloon once implanted, and structure for decoupling the delivery catheter from the balloon or balloon assembly may be included.

### CROSS-REFERENCE TO RELATED APPLICATION(S)

This application claims the benefit of the filing date of U.S. Provisional Application No. 60/449,960 filed Feb. 26, 2003, entitled METHODS AND DEVICES FOR ENDOVASCULAR MITRAL VALVE CORRECTION FROM THE LEFT CORONARY SINUS, the disclosure of which is incorporated fully herein by reference.

### FIELD

This invention relates to methods and apparatus for heart valve repair and, more particularly, to endovascular methods and apparatus for improving mitral valve function using devices inserted into the left coronary sinus.

### BACKGROUND

Mitral valve repair is the procedure of choice to correct mitral regurgitation of all etiologies. With the use of current surgical techniques, between 70% and 95% of regurgitant mitral valves can be repaired. The advantages of mitral valve repair over mitral valve replacement are well documented. These include better preservation of cardiac function and reduced risk of anticoagulant-related hemorrhage, thromboembolism and endocarditis.

In current practice, mitral valve surgery requires an extremely invasive approach that includes a chest wall incision, cardiopulmonary bypass, cardiac and pulmonary arrest, and an incision on the heart itself to gain access to the mitral valve. Such a procedure is associated with high morbidity and mortality. Due to the risks associated with this procedure, many of the sickest patients are denied the potential benefits of surgical correction of mitral regurgitation. In addition, patients with moderate, symptomatic mitral regurgitation are denied early intervention and undergo surgical correction only after the development of cardiac dysfunction.

Mitral regurgitation, or leakage from the outflow to the inflow side of the valve, is a common occurrence in patients with heart failure and a source of morbidity and mortality in these patients. Mitral regurgitation in patients with heart failure is caused by changes in the geometric configurations of the left ventricle, papillary muscles and mitral annulus. These geometric alterations result in mitral leaflet tethering and incomplete coaptation at systole. In this situation, mitral regurgitation is corrected by plicating the mitral valve annulus, either by (i) sutures alone or by (ii) sutures in combination with a support ring, so as to reduce the circumference of the distended annulus and restore the original geometry of the mitral valve annulus.

More particularly, current surgical practice for mitral valve repair generally requires that the posterior mitral valve annulus be reduced in radius by surgically opening the left atrium and then fixing sutures, or sutures in combination with a support ring, to the internal surface of the annulus; this structure is used to pull the annulus back into a smaller radius, thereby reducing mitral regurgitation by improving leaflet coaptation. This method of mitral valve repair, generally termed “annuloplasty”, effectively reduces mitral regurgitation in heart failure patients. This, in turn, reduces symptoms of heart failure, improves quality of life and increases longevity. Unfortunately, however, the invasive nature of mitral valve surgery and the attendant risks render most heart failure patients poor surgical candidates. Thus, a less invasive means to increase leaflet coaptation and thereby reduce mitral regurgitation in heart failure patients would make this therapy available to a much greater percentage of patients. Several recent developments in minimally invasive techniques for repairing the mitral valve without surgery have been introduced by several different companies. Mitralife of Santa Rosa, Calif. proposes various systems for remodeling the mitral annulus utilizing elongated structures that are percutaneously introduced into the left coronary sinus and reshape the mitral annulus therefrom. The left coronary sinus is that blood vessel commencing at the coronary ostia in the left atrium and passing through the atrioventricular groove in close proximity to the posterior, lateral and medial aspects of the mitral annulus. Because of its position adjacent the mitral annulus, the coronary sinus provides an ideal conduit for positioning an endovascular prosthesis to act on the mitral annulus and therefore re-shape it. Mitralife discloses in PCT publication WO 02/060352 and related applications a number of elongated devices that either cinch or otherwise reduce the size of the mitral annulus from the coronary sinus. Because of the complex pathway to and through the coronary sinus, the elongated devices are designed to have a first configuration for delivery and may assume a second configuration within the coronary sinus to cause reshaping of the mitral annulus. For example, an elongated tube having a natural tendency to bend is straightened with a guidewire, passed into the coronary sinus, and then the guidewire is removed to permit the tube to bend in a desired manner. Alternatively, a shape memory material may be utilized. Viacor, Inc. of Wilmington Mass. presents similar systems in PCT publication WO 02/078576, and related applications. The devices shown in the Viacor publications are primarily designed to straighten the natural curvature of at least a portion of the coronary sinus in the vicinity of the posterior leaflet of the mitral valve so that the posterior annulus displaces in an anterior direction. In one embodiment, Viacor proposes utilizing a balloon to provide the straightening force within the coronary sinus.

Despite recent attempts at minimally invasive repair of the mitral annulus using devices residing in the left coronary sinus, there is a need for such endovascular correction devices that are less traumatic to the sinus and also more reliable over the long-term. Further, there is a need for better control over the shape in which the mitral annulus is deformed by such endovascular correction devices.

### SUMMARY

The present invention provides an improved catheter-based device for reshaping a mitral valve annulus. In accordance with one embodiment of the invention, a method for endovascular mitral valve correction of a patient includes providing a catheter having a balloon assembly including a first balloon and a second balloon. The catheter includes separate lumens connected to inflate the first and second balloons. A first opposed end of the balloon assembly is flexible so as to permit bending relative to an adjacent portion of the balloon assembly. The catheter is inserted into the vasculature of the system of the patient and advanced such that the balloon assembly is located within the left coronary sinus. Subsequently, the first and second balloons are inflated.

In an alternative embodiment, the second balloon is arranged concentrically around the first balloon and has a length that is greater than the first balloon, the second balloon being mounted such that opposed ends thereof extend beyond opposed ends of the first balloon. In another alternative embodiment, the first and second balloons are arranged in series along the balloon assembly and are connected to each other by a portion of the catheter that permits relative bending therebetween.

In another embodiment of the invention, a method for endovascular mitral valve correction of a patient includes providing a catheter having a balloon on a distal end thereof, inserting the catheter into the vasculature system of the patient and advancing the catheter such that the balloon is located within the left coronary sinus. The balloon is then inflated with a fluid, and the fluid is caused to harden. The fluid can be hardened by cross-linking, e.g., injecting a cross-linking agent into the balloon or applying energy to the fluid.

In another embodiment of the invention, a system for endovascular mitral valve correction includes a catheter having a balloon assembly including a first balloon having a length and a second balloon also having a length, the catheter having separate lumens connected to inflate the first and second balloons, the balloon assembly including the first and second balloons having a total length and opposed ends, wherein at least a first opposed end of the balloon assembly is flexible so as to permit bending relative to an adjacent portion of the balloon assembly.

In yet another embodiment of the invention, a system for endovascular mitral valve correction includes a catheter having a balloon thereon having a length, the balloon having a balloon wall including a mid-section contiguous with opposed end sections, the mid-section being formed differently than at least a first end section so as to be more rigid than the first end section when the balloon is inflated.

In another embodiment of the invention, a system for endovascular mitral valve correction includes a catheter having a balloon thereon, the balloon adapted to be positioned within a coronary sinus and adapted to remodel a mitral valve annulus adjacent to the coronary sinus, the balloon containing a hardenable material to maintain the balloon in an inflated condition in the coronary sinus.

In all of the embodiments mentioned herein, the catheter may include at least one inflation lumen and one-way valve into an interior of the balloon or balloons. In addition, a mechanism may be provided for decoupling the balloon catheter from the balloon assembly in order to maintain the balloon assembly within the coronary sinus on a relatively long-term basis.

### BRIEF DESCRIPTION OF THE DRAWINGS

FIG. 1 is a schematic plan view from above of the mitral valve, left coronary sinus, and adjacent aortic valve;

FIGS. 2a and 2b are plan views of alternative mitral valve reshaping balloons of the present invention in their inflated configurations;

FIGS. 3a-3c illustrate a portion of a distal end of a catheter of the present invention having a reshaping balloon thereon and a one-way fill valve;

FIG. 4 shows the reshaping balloon of FIG. 2a deployed in the coronary sinus;

FIG. 5 shows the reshaping balloon of FIG. 2b deployed in the coronary sinus;

FIG. 6a is a schematic view of a mitral valve reshaping balloon assembly having first and second concentric balloons mounted on a catheter;

FIG. 6b is a schematic view of the balloon assembly of FIG. 6a illustrating the flexibility of opposed ends thereof;

FIGS. 7a and 7b are schematic views of, respectively, a prior art linear mitral annulus reshaper and the balloon assembly of FIG. 6a deployed in the coronary sinus;

FIG. 8a is a schematic view of a mitral valve reshaping balloon assembly having first, second, and third balloons mounted in series on a catheter;

FIG. 8b is a schematic view of the balloon assembly of FIG. 8a illustrating the flexibility of opposed ends thereof;

FIG. 9a is a schematic sectional view of the distal end of a mitral valve reshaping balloon catheter of the present invention illustrating a step of using an inflation catheter to inflate the balloon through a one-way valve; and

FIG. 9b is a schematic sectional view of the balloon catheter of FIG. 9a after removal of the inflation catheter from within the balloon assembly, thus permitting decoupling of the catheter from the reshaping balloon.

### DESCRIPTION OF THE PREFERRED EMBODIMENTS

The present application describes a number of improvements over catheter-based devices of the prior art for reshaping a mitral valve annulus by inserting the devices into the left coronary sinus. These devices are first inserted into the vascular system of the patient through a number of well-known techniques, including percutaneously via the jugular vein, and advanced through vasculature such that a distal end thereof is within the left coronary sinus. The term “distal end” to describe placement of the various annulus reshapers on the catheter is used for convenience, and a portion of the catheter or other elongated device used to position the reshaper may extend beyond the reshaper to the actual distal end of the delivery device. Therefore, “distal end” in this sense means distal along the catheter with respect to a proximal end which is outside the body. At the same time, the term catheter is representative of any elongated tube or implement that can be used to deliver the reshaping tool through the vasculature to the coronary sinus.

A number of embodiments described herein include inflation balloons for reshaping the mitral annulus. The balloons may be filled with saline or other biocompatible fluid. One aspect of the present invention is to fill the reshaping balloons with a fluid that can subsequently harden into a solid or semi-solid. For example, the fluid may cure or cross-link over time or upon exposure to heat or other stimulus. A polymeric composition such as a silicone resin, e.g., an RTV, or a 2-part epoxy resin, is suitable in this regard. Alternatively, the fluid may be of a type that can be cross-linked so as to convert from a liquid form to a solid form upon the addition of a catalyst. For example, a first fluid, such as an acrylic type resin, or a silicone resin, may be used to inflate the balloon and then a second cross-linking fluid may be added to harden the first fluid. Another potential technique included in the present invention is to harden a fluid, such as an ultraviolet light (UV) curable polymer using energy such as light delivered through a probe having a light delivery tip thereon. The probe would be advanced into proximity with the inflated balloon, or inserted to be within the interior of the balloon. These structures and techniques for converting a liquid into a solid or semi-solid will not be described in further detail herein, though it will be understood that the spectrum of such structures or techniques are covered herein.

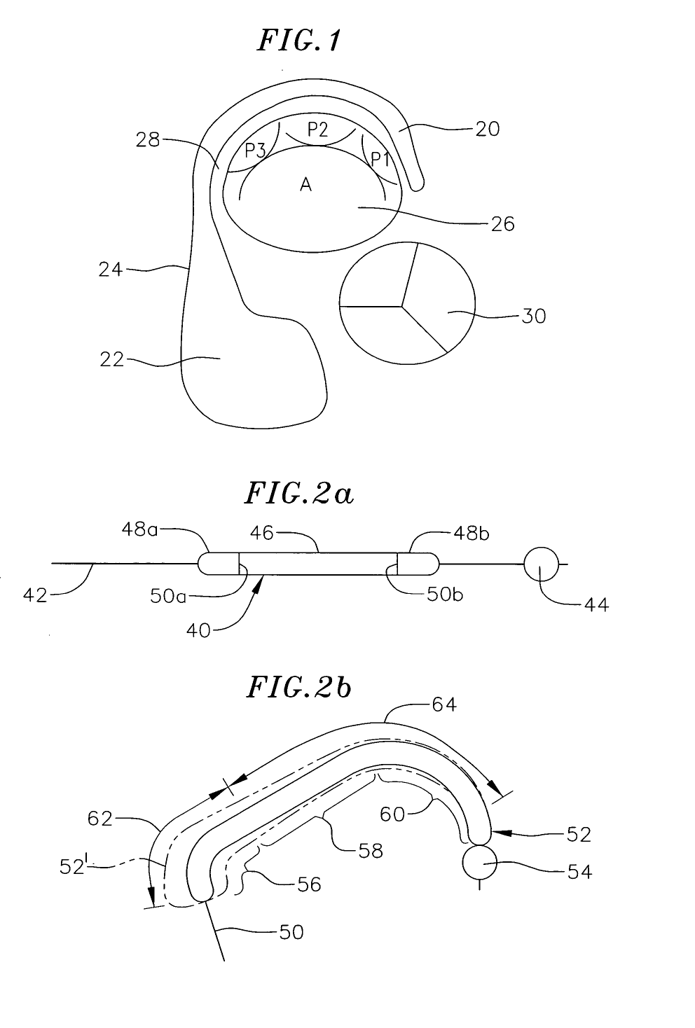

With reference to FIG. 1, the left coronary sinus 20 extends from the right atrium 22 and coronary ostia 24 and wraps around the mitral valve 26. The mitral annulus 28 is that portion of tissue surrounding the mitral valve orifice to which the several leaflets attach. The mitral valve 26 is described as having two leaflets—an anterior leaflet A and a posterior leaflet made up of three scallops P1, P2, P3. The general position of the adjacent aortic valve 30 is shown for orientation.

The problem of mitral regurgitation often results from the posterior aspect of the mitral annulus 28 dilating so as to displace one or more of the posterior leaflet scallops P1, P2, or P3 away from the anterior leaflet A. To reduce or eliminate mitral regurgitation, therefore, it is desirable to move the posterior aspect of the mitral annulus 28 in an anterior direction. For instance, in the specific case of ischemic mitral regurgitation, the posterior section of the mitral valve dilates asymmetrically, and predominantly in the region of the P3 scallop. Consequently, it is desirable to move the area of the mitral annulus 28 adjacent the P3 scallop more toward the center of the mitral valve 26 while leaving the remaining section of the mitral annulus unaltered. The catheter-based devices of the present invention can be inserted within the coronary sinus 20 to the proper location so as to perform the desired reshaping procedure on the mitral annulus 28.

FIG. 2a illustrates a first embodiment of a catheter-based mitral annulus-reshaping device of the present invention that includes a single balloon 40 mounted on or near the distal end of a catheter 42. If desired, a second balloon 44 for anchoring the reshaping balloon 40 within the coronary sinus 20 may be provided, typically distally on the catheter with respect to the reshaping balloon, as shown. The reshaping balloon 40 comprises a mid-section 46 contiguous with a pair of opposed end sections 48a, 48b. Continuous in this sense means that the balloon 40 has a single-layered balloon wall along its entire length such that the mid-section 46 and end sections 48a, 48b define a continuous tube connected at joint lines 50a, 50b. The mid-section 46 has a different construction than the end sections 48a, 48b so as to be more rigid when the balloon 40 is inflated. For example, the mid-section 46 may have a greater wall thickness than the end sections 48a, 48b, or may be impregnated with a tubular matrix of fabric or the like. Alternatively, the mid-section 46 may be a different material than the end sections 48a, 48b, the two materials being joined at the lines 50a, 50b such as with co-extrusion, adhesives, or ultrasonic welds. A combination of polymers such as nylon, PEBAX, PET, polyethylene (different durometers) can also be used, such as by laminating or co-extruding the multiple materials. When the balloon 40 is inflated, the mid-section 46 provides a relatively rigid, linear reshaping portion while the end sections 48a, 48b are capable of flexing or bending to conform to the curvilinear coronary sinus 20 and reduce damage thereto.

FIG. 2b illustrates a second embodiment of a mitral annulus-reshaping device having a catheter 50 on which a hook-shaped or otherwise curvilinear balloon 52 is mounted. The balloon 52 is delivered in a deflated state and assumes the illustrated shape upon inflation. Again, a second, typically smaller anchoring balloon 54 may be utilized in conjunction with the reshaping balloon 52. In the specific embodiment illustrated, the reshaping balloon 52 includes a curvilinear proximal end section 56, a generally linear mid-section 58, and a curvilinear distal end section 60. The end sections 56, 60 may have different shapes, for example, the proximal end section 56 is shorter than the distal end section 60. Likewise, the mid-section 58 may be other than linear, and there may be more than three discrete sections along the reshaping balloon 52. It will therefore be appreciated that an inflatable reshaping device can take an infinite number of forms and may be customized to correct particular pathologies within the diseased mitral valve.

Desirably, the balloon 52 when inflated is tapered as depicted in phantom at 52′ in FIG. 2b such that the proximal end section 56 has a greater outer diameter (OD) than the distal end section 60. On average, the coronary sinus inner diameter (ID) tapers down from about 15 mm at the proximal coronary ostia to about 5 mm at the distal end. The OD of the balloon 52 should therefore match (or at least correlate with) the sinus ID at the implant location. The balloon 52 may be undersized slightly to permit blood flow there around. Therefore, for example, a balloon 52 that extends the entire length may have an OD of about 15 mm at the proximal end section 56 and an OD of about 5 mm at the distal end section 60. In other exemplary configurations, shorter balloons may taper from 6-4 mm OD, or from 15-10 mm OD, depending on the placement. These diameter ranges apply to all of the embodiments in the present application. In the illustrated embodiment, a proximal length 62 of the balloon 52′ has a relatively steep taper while a distal length 64 has a more gradual taper. This configuration is believed to most accurately mimic the average coronary sinus shape.

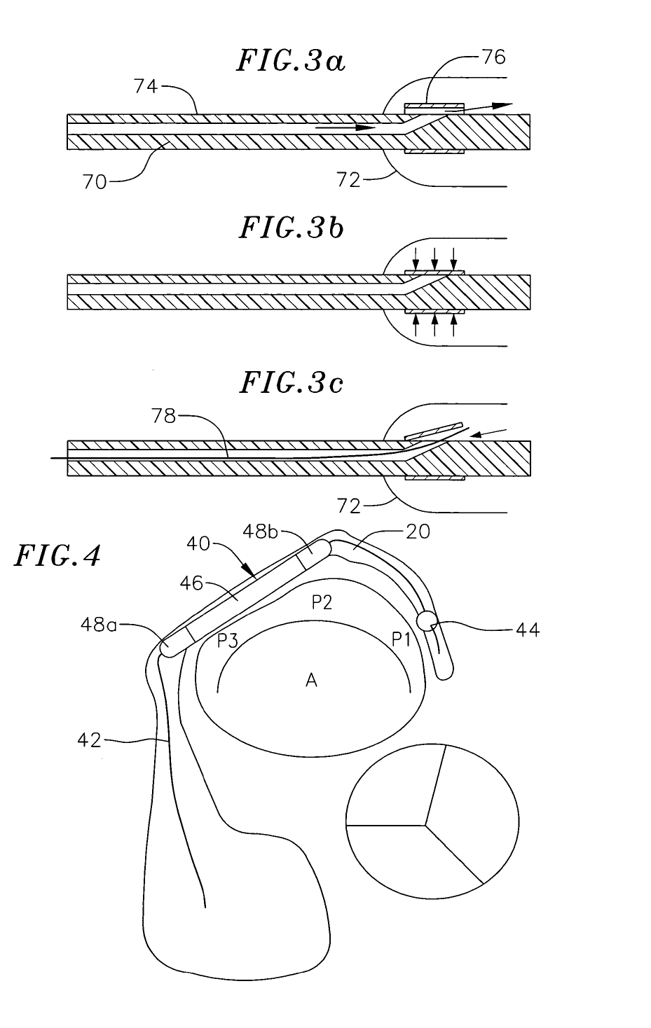

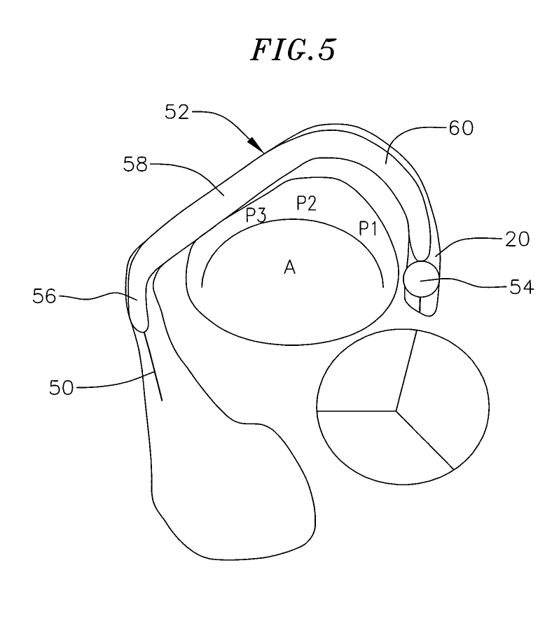

FIGS. 3a-3c illustrate a particular configuration during several steps of inflation of any of the balloons of the present invention. A balloon catheter 70 carries the reshaping balloon 72 on its distal end. An inflation lumen 74 opens through a one-way valve 76 into the interior of the balloon 72. The one-way valve 76 in the illustrated embodiment comprises an elastic sleeve that is easily displaced radially outward upon fluid pressure within the lumen 74, as shown in FIG. 3a. The balloon 72 may be made from high-pressure resistant polymer, such as polyethylene terephthalate (PET), which is capable of the withstanding pressures of up to 400 psi. When inflated to such pressures, the balloon 72 becomes relatively rigid and maintains its preformed shape. Also, the high-pressure within the balloon 72 acts on the exterior surface of the one-way valve 76, as seen in FIG. 3b, and prevents deflation of the balloon. In case of complications, or other need to deflate the balloon 72, a guidewire 78 or other such probe may be passed through the lumen 74 as seen in FIG. 3c so as to displace the one-way valve 76 outward or puncture the one way valve 76 and relieve pressure from within the balloon 72.

FIG. 4 illustrates the placement of the mitral annulus-reshaping device of FIG. 2a, wherein the reshaping balloon 40 is inflated on or near the distal end of the catheter 42 in a predetermined position within the coronary sinus 20. Specifically, the relatively rigid linear mid-section 46 of the balloon 40 is generally centered in the coronary sinus next to the P3 scallop of the posterior leaflet. This placement is particularly beneficial for correcting ischemic mitral regurgitation. The relatively more flexible end sections 48a, 48b reduce potential damage or other trauma to the walls of the coronary sinus 20.

The anchoring balloon 44 is shown further along the coronary sinus from the reshaping balloon 40. Because the operation is carried out while the heart is beating, the anchoring balloon 44 helps prevent migration of the reshaping balloon 46 prior to its inflation. The diameter of the reshaping balloon 40 is such that blood is permitted to flow around it after inflation. Alternatively, longitudinal channels or grooves can be provided in the balloon 40 to permit greater blood flow. For example, one or more spiral grooves or channels may be formed in the exterior of the balloon 40. In a further alternative, the balloon 40 may completely occlude the coronary sinus 20 such that blood no longer flows therethrough. Some studies indicate that collateral perfusion of the heart in the absence of flow through the coronary sinus 20 is sufficient.

FIG. 5 illustrates the placement of the curvilinear mitral annulus-reshaping device of FIG. 2b, wherein the reshaping balloon 52 is inflated on or near the distal end of the catheter 50 within the coronary sinus 20. Again, the relatively linear mid-section 58 is positioned adjacent the P3 scallop of the posterior leaflet to correct for ischemic mitral regurgitation. The curvilinear end sections 56, 60 are shown conforming to the curvature of the coronary sinus 20, which helps reduce trauma thereto. Also, the anchoring balloon 54 is seen inflated just distal to the reshaping balloon 52. As before, the anchoring balloon 54 is typically deployed prior to inflation of the reshaping balloon 52.

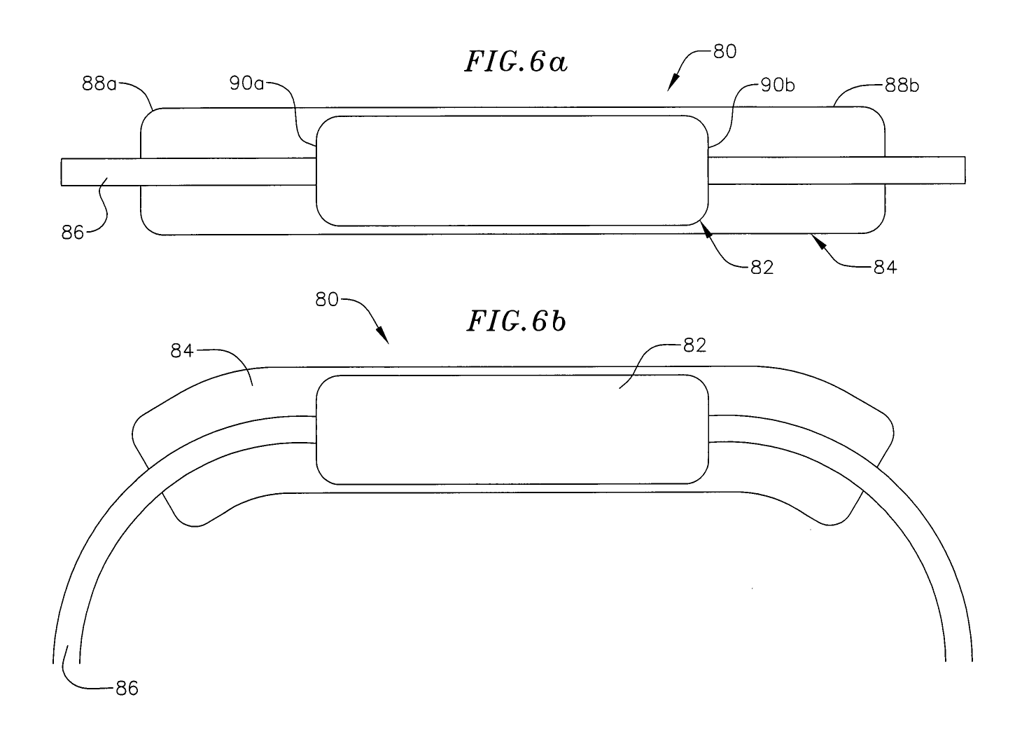

FIGS. 6a and 6b schematically illustrate a still further catheter-based reshaping device of the present invention. A balloon assembly 80 comprising a first balloon 82 and a second balloon 84 mounts at or near the distal end of a balloon catheter 86. Although not shown, the balloon catheter 86 includes one or more lumens for jointly or independently inflating the balloons 82, 84. The second balloon 84 is arranged concentrically around the first balloon 82 and has a length that is greater than the first balloon. In the illustrated embodiment, the second balloon 84 is mounted such that opposed ends 88a, 88b thereof extend beyond opposed ends 90a, 90b of the first balloon 82. Consequently, the opposed ends 88a, 88b of the second balloon 84 define the opposed ends of the balloon assembly. The length of the balloon assembly 80 is desirably at least about one-third the length of the coronary sinus, or at least the length between the commissures of the adjacent mitral valve, transferred to the coronary sinus. In an exemplary embodiment, the length of first balloon 82 may be between about 50-100 mm, and the second balloon 84 may be sized longer such that the opposed ends 88a, 88b overhang the first balloon by about 10 mm. Another way to look at the length ranges is that the opposed ends 88a, 88b each has a length that is between about 8-17% of the total length of the balloon assembly 80 (both ends 88a, 88b and a first balloon 82 having a length of 100 mm, versus one end 88a and a first balloon 82 having a length of 50 mm). These length ranges apply to all of the embodiments in the present application.

Because of different materials or construction, or because the second balloon 84 is inflated to a lesser pressure than the first balloon 82, the opposed ends of the balloon assembly 80 are relatively more flexible than a mid-section thereof. The inner or first balloon 82 may be inflated to between about 3-20 atmospheres, while the second balloon 84 is inflated to between about 1-6 atmospheres. More specifically, the mid-section of the balloon assembly 80 comprises that portion coincident with the first, inner balloon 82, and the opposed ends of the balloon assembly coincide with the opposed ends 88a, 88b of the second balloon 84. FIG. 6b illustrates the balloon assembly 80 after inflation and after deployment within the coronary sinus (not shown), or when subjected to bending stresses. The opposed ends of the balloon assembly 80 are permitted to flex to help prevent undue trauma to the inner walls of the coronary sinus. Prior to implant, the balloon assembly 80 is desirably deflated and flattened by pulling a vacuum on both balloons 82, 84 and then wrapped and heat set within a tube to create a low delivery profile.

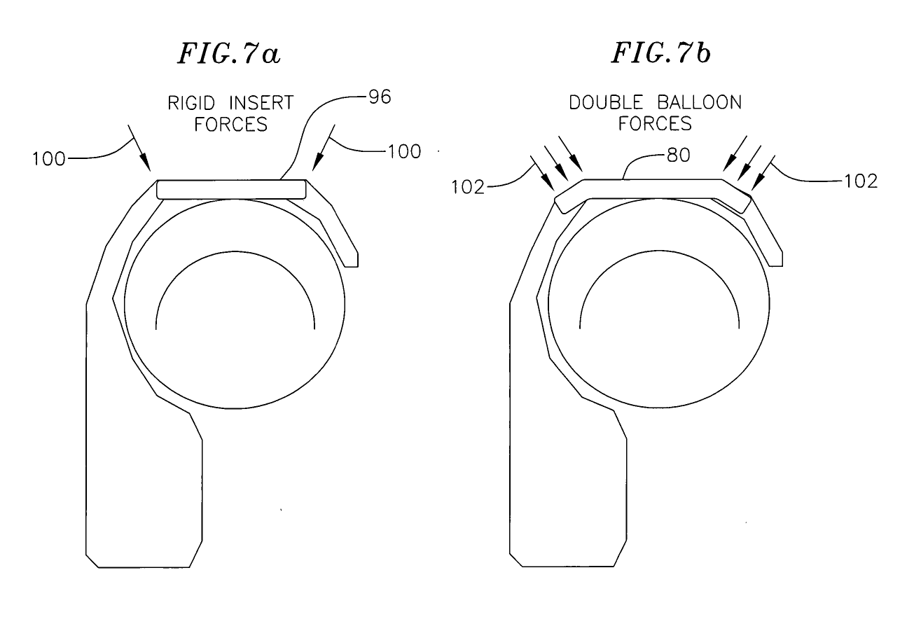

FIGS. 7a and 7b contrast the deployment of a linear, rigid insert 96 (FIG. 7a) in the coronary sinus and a mitral valve reshaping device, such as balloon assembly 80 of FIG. 6a (FIG. 7b). FIG. 7a illustrates the reaction forces 100 applied by the outer curve of the coronary sinus on the ends of the rigid insert 96. This concentration of reaction forces 100 tends to create points of abrasion or trauma within the coronary sinus. On the other hand, FIG. 7b illustrates reaction forces 102 that are more widely distributed along the opposed ends of the balloon assembly 80. Further, the opposed ends of the balloon assembly 80 are relatively flexible and curve to conform to coronary sinus, therefore avoiding altering the adjacent mitral annulus.

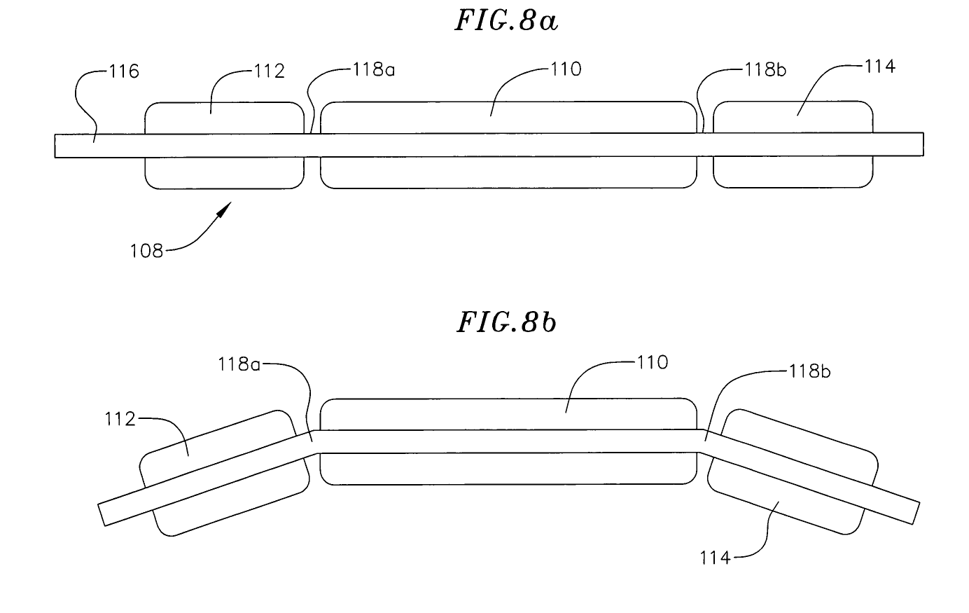

FIGS. 8a and 8b illustrate a still further embodiment of a mitral valve reshaping device of the present invention. In particular, a balloon assembly 108 comprises a first balloon 110, a second balloon 112, and a third balloon 114 mounted in series along a balloon catheter 116. The first balloon 110 is located in between the second and third balloons 112, 114. Small gaps remain between the balloons 110, 112, 114 so that short portions 118a, 118b of the catheter 116 permit bending of the balloons relative to each other, as seen in FIG. 8b. In this manner, the opposed ends of the balloon assembly 108 are rendered more flexible than a mid-section thereof, in the same manner as the concentric balloon assembly 80 of FIGS. 6a and 6b. The deployment of the balloon assembly 108 in the coronary sinus therefore results in less trauma to the surrounding tissue.

Preferably, the central, first balloon 110 is substantially linear and has a length greater than either of the second or third balloons 112, 114. Also, the balloon assembly 108 may be provided with only one end balloon 112 or 114, depending on the need. As mentioned above, the balloon catheter 116 may be provided with a single inflation lumen for simultaneously inflating the three balloons 110, 112, 114, or separate inflation lumens may be utilized. If separate lumens are utilized, one or more of the series of balloons, typically the central balloon 110, may be inflated to a greater pressure than the others to provide a more rigid reshaping force at that location.

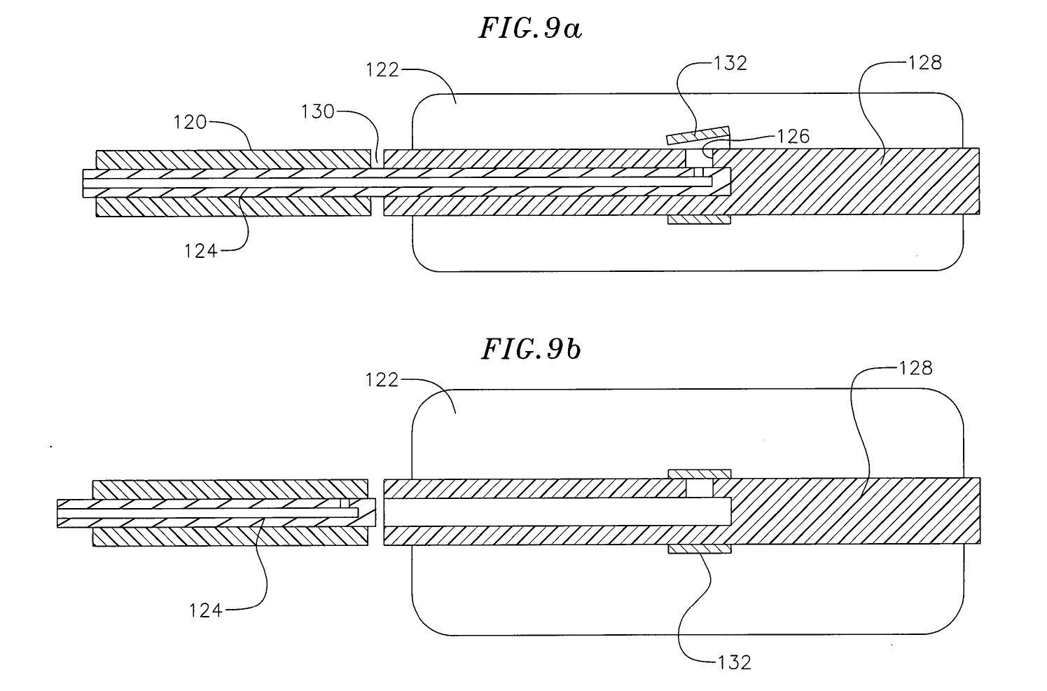

FIGS. 9a and 9b illustrate structure and use of an exemplary mechanism for decoupling a balloon catheter 120 from a mitral valve reshaping balloon assembly 122. The various devices of the present invention are intended to be implanted within the coronary sinus on a relatively long-term basis. Therefore, the delivery catheter must be removed and a mechanism for decoupling the two provided.

In the illustrated embodiment, an inflation catheter 124 extends through a lumen of the balloon catheter 120 and terminates adjacent an inflation port 126 formed in a distal extension 128 of the balloon catheter. The distal extension 128 is coextensive with, but decoupled from, the main length of the balloon catheter 120, as seen at gap 130. A one-way valve 132, such as the elastic sleeve described above, cooperates with inflation port 126 to permit inflation of the balloon assembly 122 and prohibit deflation thereof. The distal end of the inflation catheter 124 fits closely within the lumen of the extension 128, or is otherwise temporarily secured thereto during delivery and deployment of the balloon assembly 122. After inflation of the balloon assembly 122, the inflation catheter 124 is removed from within the distal extension 128, such as by proximally withdrawing the inflation catheter while holding the balloon catheter 120 stationery. Once the inflation catheter 124 is completely withdrawn from the distal extension 128, the balloon assembly 122 with the distal extension are decoupled from the proximal portion of the balloon catheter 120. Of course, other arrangements for performing the decoupling function are contemplated within the scope of present invention.

While the foregoing describes the preferred embodiments of the invention, various alternatives, modifications, and equivalents may be used. Moreover, it will be obvious that certain other modifications may be practiced within the scope of the appended claims.

What is claimed is:

1. A method for endovascular mitral valve correction of a patient, comprising:
providing a catheter having a balloon assembly including a first balloon having a length and a second balloon also having a length, the catheter having separate lumens connected to inflate the first and second balloons, the balloon assembly including the first and second balloons having a total length and opposed ends, wherein at least a first opposed end of the balloon assembly is flexible so as to permit bending relative to an adjacent portion of the balloon assembly;
inserting the catheter into the vasculature system of the patient;
advancing the catheter such that the balloon assembly is located within the left coronary sinus; and
inflating the first and second balloons.

2. The method of claim 1, wherein the second balloon is arranged concentrically around the first balloon and has a length that is greater than the first balloon, the second balloon being mounted such that opposed ends thereof extend beyond opposed ends of the first balloon and define the opposed ends of the balloon assembly.

3. The method of claim 2, further including:
inflating the first balloon to a first pressure; and
inflating the second balloon to a second pressure less than the first such that the opposed ends of the second balloon that extend beyond the opposed ends of the first balloon render the opposed ends of the balloon assembly relatively more flexible than a mid-portion thereof.

4. The method of claim 1, wherein the first and second balloons are arranged in series along the balloon assembly and are connected to each other by a portion of the catheter that permits relative bending therebetween so that the second balloon defines the first opposed end of the balloon assembly that is flexible.

5. The method of claim 4, further including a third balloon arranged in series adjacent the first balloon along the balloon assembly such that the first balloon is between the second and third balloons, the third balloon being connected to the first balloon by a portion of the catheter that permits relative bending therebetween so as to define a second opposed end of the balloon assembly that is flexible.

6. The method of claim 5, wherein the second and third balloons each have a length that is shorter than the length of the first balloon.

7. The method of claim 1, wherein prior to inserting the catheter into the vasculature system of the patient the balloon assembly is deflated and wrapped and heat set within a tube to create a low delivery profile.

8. A method for endovascular mitral valve correction of a patient, comprising:
providing a catheter having a balloon on a distal end thereof;
inserting the catheter into the vasculature system of the patient;
advancing the catheter such that the balloon is located within the left coronary sinus;
inflating the balloon with a fluid; and
causing the fluid to harden.

9. The method of claim 8, wherein the step of causing the fluid to harden comprises cross-linking the fluid.

10. The method of claim 9, wherein the step of cross-linking the fluid is accomplished by an action selected from the group consisting of:
injecting a cross-linking agent into the balloon; and
applying energy to the fluid.

11. The method of Claim 8, wherein the step of causing the fluid to harden comprises curing the fluid.

12. The method of claim 8, wherein the balloon after being inflated with the fluid has a non-linear shape.

13. The method of claim 8, wherein prior to inserting the catheter into the vasculature system of the patient the balloon assembly is deflated and wrapped and heat set within a tube to create a low delivery profile.

14. A system for endovascular mitral valve correction, comprising:
a catheter having a balloon assembly including a first balloon having a length and a second balloon also having a length, the catheter having separate lumens connected to inflate the first and second balloons, the balloon assembly including the first and second balloons having a total length and opposed ends, wherein at least a first opposed end of the balloon assembly is flexible so as to permit bending relative to an adjacent portion of the balloon assembly.

15. The system of claim 14, wherein the second balloon is arranged concentrically around the first balloon and has a length that is greater than the first balloon, the second balloon being mounted such that opposed ends thereof extend beyond opposed ends of the first balloon and define the opposed ends of the balloon assembly.

16. The system of claim 14, wherein the first and second balloons are arranged in series along the balloon assembly and are connected to each other by a portion of the catheter that permits relative bending therebetween so that the second balloon defines the first opposed end of the balloon assembly that is flexible.

17. The system of claim 14, further including a third balloon arranged in series adjacent the first balloon along the balloon assembly such that the first balloon is between the second and third balloons, the third balloon being connected to the first balloon by a portion of the catheter that permits relative bending therebetween so as to define a second opposed end of the balloon assembly that is flexible.

18. The system of claim 17, wherein the second and third balloon each has a length that is shorter than the length of the first balloon.

19. The system of claim 14, wherein the first opposed end that is flexible has a length that is between about 8-17% of the total length of the balloon assembly.

20. The system of claim 14, wherein the balloon assembly is tapered when inflated such that a proximal end has a greater outer diameter than a distal end thereof.

21. A system for endovascular mitral valve correction, comprising:
a catheter having a balloon thereon having a length, the balloon having a balloon wall including a mid-section contiguous with opposed end sections, the mid-section being formed differently than at least a first end section so as to be more rigid than the first end section when the balloon is inflated.

22. The system of claim 21, wherein the mid-section has a greater wall thickness than the first end section.

23. The system of claim 21, wherein the mid-section is impregnated with a tubular matrix.

24. The system of claim 21, wherein the mid-section is formed of a different material than the first end section and joined thereto at bond lines.

25. The system of claim 21, wherein the first end section has a length that is between about 8-17% of the total length of the balloon.

26. The system of claim 21, wherein the balloon is tapered when inflated such that a proximal end has a greater outer diameter than a distal end thereof.

27. The system of claim 14, wherein the catheter includes at least one inflation lumen and one-way valve into an interior of the first and second balloons.

28. The system of claim 21, wherein the catheter includes an inflation lumen and one-way valve into an interior of the balloon.

29. A system for endovascular mitral valve correction comprising:
a catheter having a balloon thereon, the balloon adapted to be positioned within a coronary sinus and adapted to remodel a mitral valve annulus adjacent to the coronary sinus, the balloon containing a hardenable material to maintain the balloon in an inflated condition in the coronary sinus.

30. The system of claim 29 wherein the hardenable material is at least one of an acrylic resin, a silicone resin and an ultraviolet light curable material.
